(19) United States
(12) Patent Application Publication
(10) Pub. No.: US 2010/0030337 A1
(43) Pub. Date: Feb. 4, 2010

(54) Spine Surgery Technique And System

(76) Inventors: Nasser Ani, Holmdel, NJ (US); Darren Freeman, (US)

Correspondence Address: RAYMOND R. MOSER JR., ESQ.;MOSER IP LAW GROUP, 1030 BROAD STREET, 2ND FLOOR, SHREWSBURY, NJ, 07702, US

(21) Appl. No.: 12/185,415

(22) Filed: Aug. 4, 2008

(51) Int. Cl. A61F 2/44; A61B 19/00; A61B 17/04

(52) U.S. Cl. 623/17.16; 128/898; 606/300

(57) ABSTRACT

A system and method for use in spinal surgery to alleviate a bulging/herniated disc. The method comprises inserting a first cannula into a vertebra, and inserting a first anchor into the first cannula through the vertebra and into a disc, the first anchor generating a magnetic field pointing in a first direction. The method further comprises inserting a second anchor through the first cannula and through the vertebra, the second anchor generating a magnetic field pointing in the first direction, the second anchor including a second cannula; and pulling the second cannula to anchor the second anchor into the vertebra. As the magnetic fields are in the same direction, the first anchor repels the second anchor thereby pushing a bulging disc back in place.

### BACKGROUND OF THE INVENTION

1. Field of the Invention

This disclosure relates to a spine surgery technique and, more particularly, to a technique that uses magnets and magnetic fields to alleviate a bulging disc, herniated disc or degenerative disc of the spine or any associated pain.

2. Description of the Related Art

Referring to FIG. 1, a spine 50 typically comprises a plurality of vertebrae 52, 54 stacked on top of one another. The vertebrae protect a spinal cord 56. In between vertebrae 52, 54 are discs 60. Discs 60 act as a cushion between adjacent vertebrae 52, 54 and allow for comfortable relative movement of vertebrae 52, 54. Spinal cord 56 branches off into a plurality of nerves 58. Nerves 58, in turn, are disposed within tunnels (not explicitly shown), such as the foraminal canal, and travel to various parts of the body.

Disc 60 can thought of as a donut with a plurality of concentric rings. A center of disc 60 includes sponge-like material (nucleus pulposus) which provides the cushioning. Over the course of time, or due to injury, inner rings of disc 60 may crack causing the nucleus pulposus to flow through the crack and create a bulge on one side of the disc. That bulge may protrude into the tunnel where nerve 58 is disposed, may enter into a spinal canal where spinal cord 56 is disposed, or even traverse outside of a normal perimeter of spine 50. Commonly, the bulge goes into the foramina canal and causes stenosis or a narrowing of that canal. If left untreated, this condition may cause back pain, leg pain and radicular pain and may result in continued degeneration of the disc.

Prior art attempts for handling this bulging disc problem are largely unsuccessful. Some techniques include removing a piece of bone in an adjacent vertebra. Such a procedure is painful, requires long recuperation time, may cause instability in the joint between adjacent vertebrae, and does not necessarily prevent the disc from herniating again. The bulge itself can be cut and removed but the disc cannot simply be tied off to prevent the nucleus pulposus from further leaking out and/or causing further herniation. Some prior art techniques remove a portion of the nucleus pulposus in the disc but this results in a less flexible disc. Such a disc may decompress and further degenerate resulting in little to no cushion between vertebrae.

One prior art technique is shown in U.S. Patent publication 2006/0247782. In that publication, a patient's disc is removed and replaced with a prosthetic disc having distal ends including electromagnets. A magnetic field is created between the magnets to repel one end portion of the prosthetic disc from the other end portion, creating a simulated cushion. The use of such a device and technique requires physical removal of a patient's disc necessitating significant back surgery and recovery time and introducing possible complications relating to rejection of the prosthetic.

### SUMMARY OF THE INVENTION

One embodiment of the invention is a method for performing spine surgery. The method comprises inserting a first anchor into a disc, the first anchor generating a first magnetic field pointing in a first direction, the disc being between a first and a second vertebra. The method further comprises inserting a second anchor into the first vertebra, the second anchor generating a second magnetic field pointing in the first direction.

Another embodiment of the invention is a method for performing spine surgery. The method comprises inserting a first cannula into a vertebra and inserting a first anchor into the first cannula through the vertebra and into a disc, the first anchor generating a first magnetic field pointing in a first direction. The method further comprises inserting a second anchor through the first cannula and through the vertebra, the second anchor generating a second magnetic field pointing in the first direction; and pulling the second anchor into the vertebra.

Yet another embodiment of the invention is a system for use in spine surgery. The system comprises a first anchor, the first anchor including a first base, first and second arms pivotably connected to the first base, the first and second arms generating a first magnetic field pointing in a first direction. The system further comprises a second anchor, the second anchor including a second base, and third and fourth arms pivotably connected to the second base, the third and fourth arms generating a second magnetic field pointing in the first direction.

### BRIEF DESCRIPTION OF THE DRAWINGS

The drawings constitute a part of the specification and include exemplary embodiments of the present invention and illustrate various objects and features thereof.

FIG. 1 is a front cut-away view of a spine in accordance with the prior art.

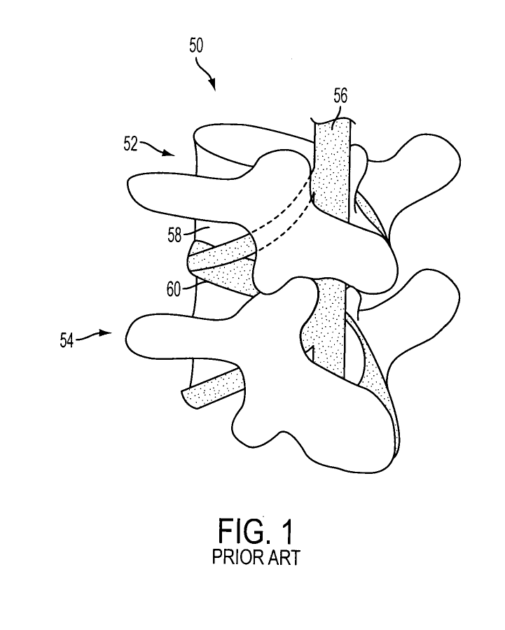

FIG. 2 is a front cut-away view of a spine and illustrating a disc surgery technique in accordance with an embodiment of the invention.

FIG. 3 is a top view of an anchor which may be used in a disc surgery technique in accordance with an embodiment of the invention.

FIG. 4 is a top view of an anchor inserted into a cannula in accordance with an embodiment of the invention.

FIG. 5 is a top view of an anchor inserted into a cannula and a disc in accordance with an embodiment of the invention.

FIG. 6 is a top view of an anchor inserted into a disc in accordance with an embodiment of the invention.

FIG. 7 is a top view of an anchor inserted into a cannula and advanced through a bone in accordance with an embodiment of the invention.

FIG. 8 is a top view of an anchor inserted into a cannula and a bone in accordance with an embodiment of the invention.

FIG. 9 is a top view of an anchor inserted past a bone in accordance with an embodiment of the invention.

FIG. 10 is a top view of an anchor pulled back onto a bone and a screw in accordance with an embodiment of the invention.

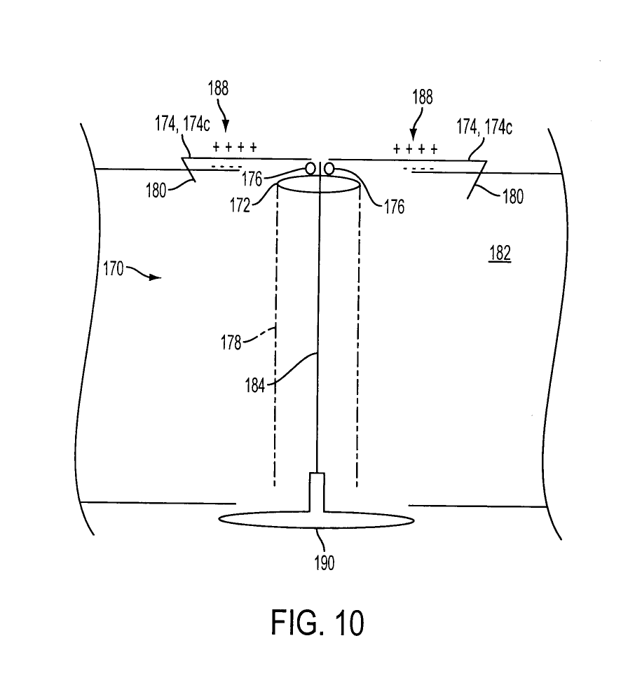

FIG. 11 is a top view of a disc, a vertebra and two anchors in accordance with an embodiment of the invention.

### DETAILED DESCRIPTION OF THE PREFERRED EMBODIMENT(S)

Various embodiments of the invention are described hereinafter with reference to the figures. Elements of like structures or function are represented with like reference numerals throughout the figures. The figures are only intended to facilitate the description of the invention as a limitation on the scope of the invention. In addition, an aspect described in conjunction with a particular embodiment of the invention is not necessarily limited to that embodiment and can be practiced in conjunction with any other embodiments of the invention.

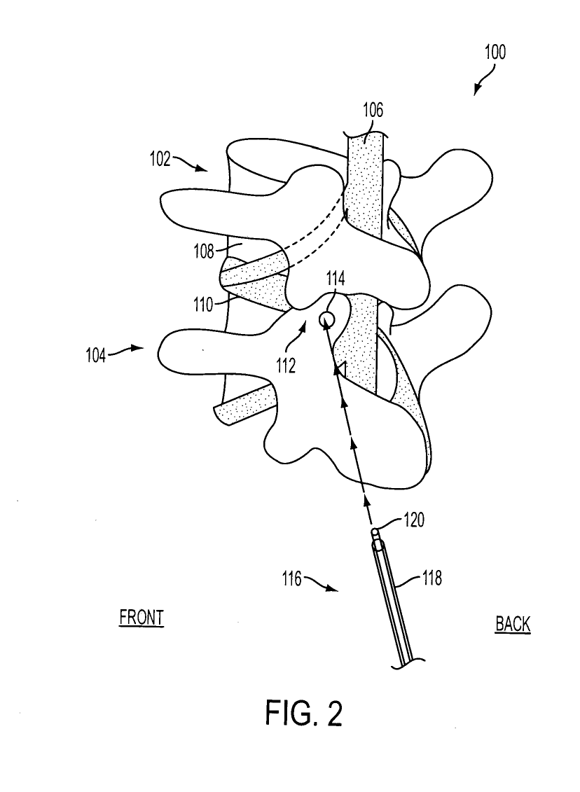

Referring now to FIG. 2, there is shown a spine 100 upon which a technique in accordance with the invention may be used. Spine 100 comprises a plurality of vertebrae 102, 104 stacked on top of one another. The vertebrae protect a spinal cord 106. In between vertebrae 102, 104 are discs 110. Discs 110 act a cushion between adjacent vertebrae 102, 104 and allow for comfortable relative movement of vertebrae 102, 104. Spinal cord 106 branches off into a plurality of nerves 108. Nerves 108, in turn, are disposed within tunnels (not explicitly shown).

Unlike prior art techniques, the embodiments shown allow a physician to generally maintain a patient's anatomy and basically push a bulging/herniated disc back in place. As shown, a cannula 116 is used to bore a hole through vertebra 104 up to disc 110. Cannula 116 includes an outer sleeve 118 and an inner sleeve 120. Under fluoroscopic guidance, cannula 116 is inserted into a patient from the posterior (back) side of the patient toward the anterior (front) side. The insertion is performed near a facet joint 112 and forms an opening 114. Bulges in discs 110 are frequently formed near facet joint 112. A tip of inner sleeve 120 may be pointed so that cannula 116 may be inserted into a patient manually or with a hammer. An inner diameter of cannula 116 may be, for example, 2.5 mm. Cannula 116 is passed over a guide wire (not shown).

Cannula 116 is inserted through vertebra 104 but not into disc 110. A hollow knob (not shown) may be circumferentially placed over cannula 116 and tightened on the patient's skin so as to secure cannula 116 to the patient. Inner sleeve 120 is removed from outer sleeve 118. A circular drill bit (not shown) is inserted circumferentially over the guide wire and through outer sleeve 118. The circular drill bit enlarges hole 114 and creates jagged edges in hole 114 facilitating growth of bone on an inserted cannula as is discussed below. The drill bit and guide wire are then removed.

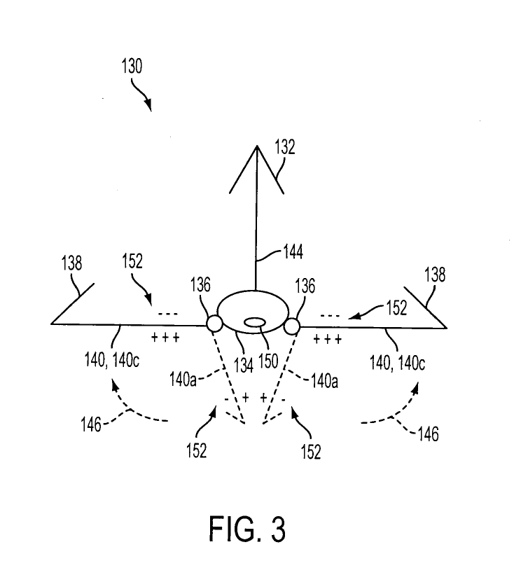

FIG. 3 shows a first anchor 130 which may be used in accordance with an embodiment of the invention. First anchor 130 includes a base 134, a body 144, a hooked end 132 and arms 140. Arms 140 may be, for example, permanent magnets and 2 mm long. Base 134 may have a sharp edge to facilitate secure placement of first anchor 130. Base 134 includes hinges 136 allowing arms 140 to pivotably move with respect to base 134. Hinges 136 bias arms 140 toward a position that is substantially perpendicular to body 144 as is shown at position 140c in FIG. 3. Hinges 136 also allow arms 140 to be positioned obliquely to body 144 as shown in phantom at position 140a. Arrows 146 indicate the movement of arms 140 from position 140a to position 140c caused by the bias of hinges 136. Arms 140 may initially be disposed almost parallel to body 144 as shown at position 140a in FIG. 3. As first anchor 130 is inserted into a disc (explained in more detail below) arms 140 move to position 140c because of the bias of hinges 136. First anchor 130 also includes tines 138 disposed oblique to arms 140a. Tines 138 may be, for example, 1-2 mm long.

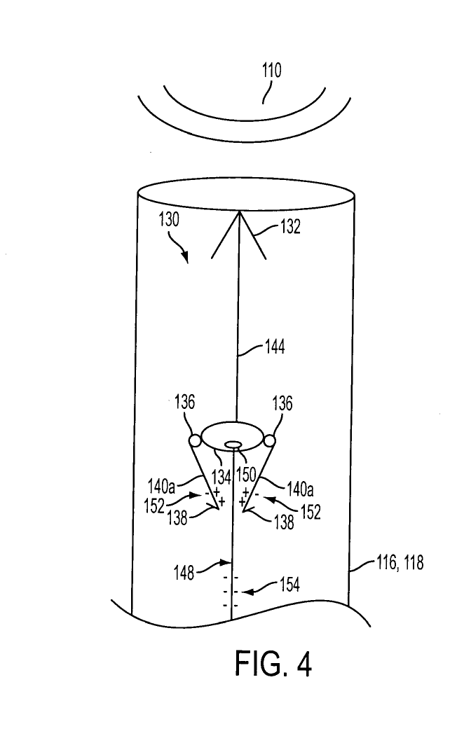

Referring now to FIGS. 3 and 4, first anchor 130 may be inserted into a disc 110 in the following manner. An introducer 148 is connected with first anchor 130 at notch 150 (best seen in FIG. 3) of first anchor 130. The combination of introducer 148 and first anchor 130 is then inserted into outer sleeve 118 of introducer 116. Arms 140 of first anchor 130 are designed to generate a magnetic field pointing in a first direction—illustrated by polarity marker 152 in the figures. Introducer 148 is designed to generate a magnetic field pointing in a second direction opposite to that of arms 140—as is illustrated with polarity marker 154 in the figures. Introducer 148 thus attracts arms 140. Introducer 148 may be, for example, made of a ferromagnetic material such as titanium. The combination of the walls of outer sleeve 118 and the magnetic attraction between arms 140 and introducer 148 causes arms 140 to move backward against the bias of hinge 136 and to assume position 140a.

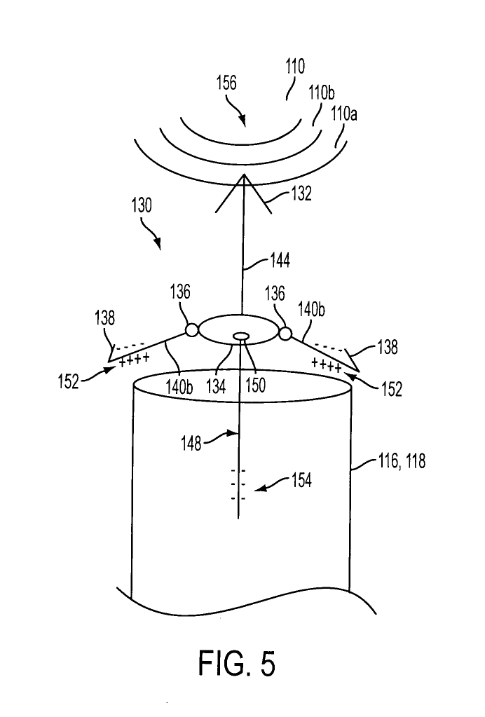

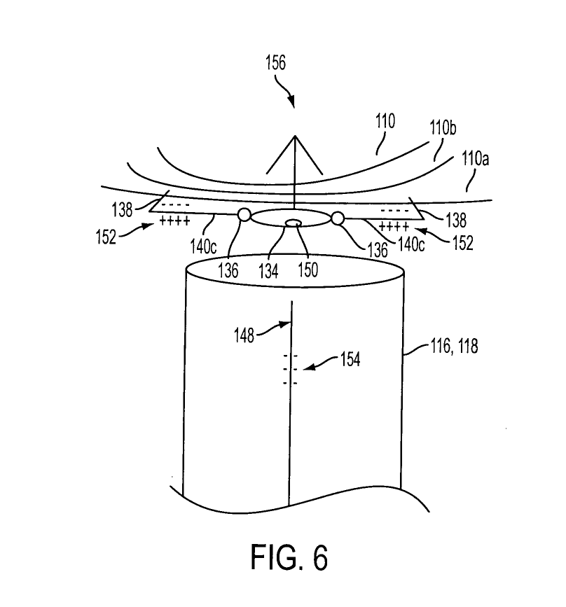

Referring to FIGS. 3 and 5, a physician may continue to push the combination of introducer 148 and first anchor 130 so that hooked end 132 penetrates the outer rings 10a, 10b or annulus of disc 110 and travels toward a nucleus 156 of disc 110. Once base 134 reaches disc 110, introducer 148 may be detached from first anchor 130 and withdrawn from cannula 116. As shown in FIG. 5, when introducer 148 is detached from first anchor 130 and arms 138 are no longer proximate to the attractive magnetic field generated by introducer 148, hinge 136 moves arms 140 in the direction indicated by arrows 146 toward position 140b. Hinge 136 continues to move arms 140 until arms 140 are disposed in position 140c and shown in FIG. 6. In position 140c, tines 138 enter disc 110 and anchor first anchor 130 to disc 110. Disc 110 then collapses on tines 138. Introducer 148 is thereafter removed completely from outer sleeve 118.

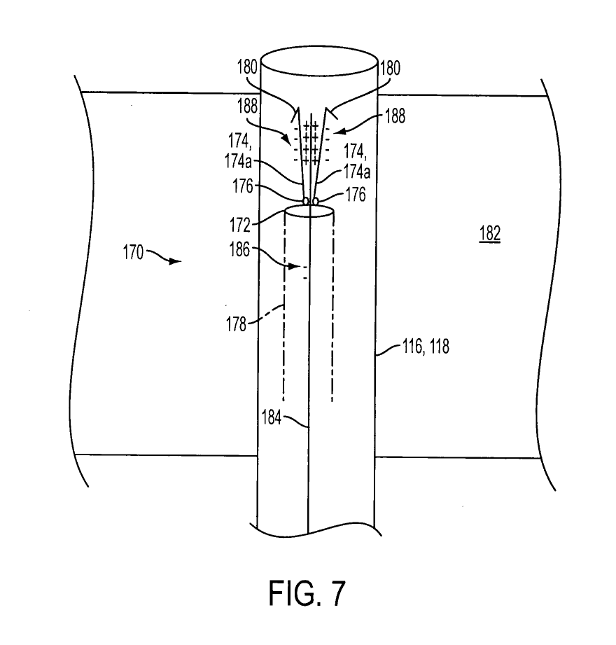

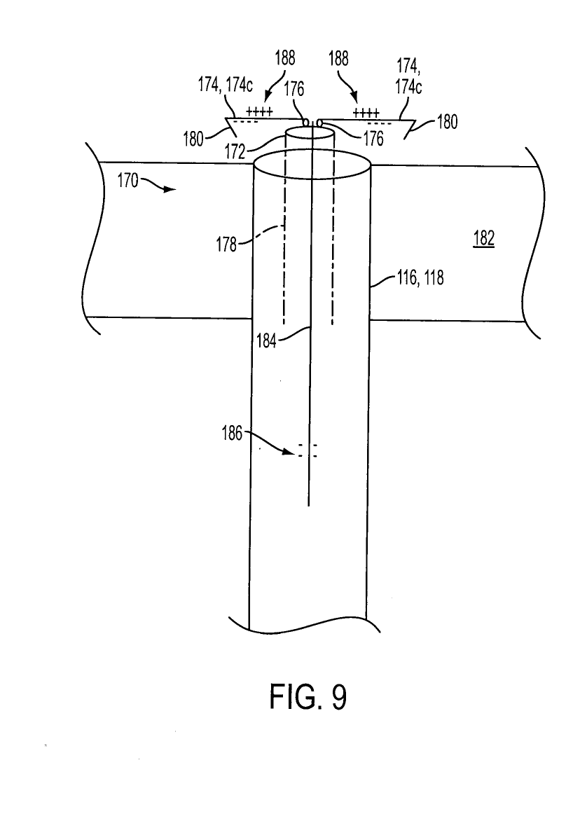

A second anchor may now be placed in a vertebra proximate to first anchor 130 and disc 110. Referring to FIG. 7, there is shown a second anchor 170 which may be used in accordance with an embodiment of the invention. Second anchor 170 includes a base 172, arms 174 and an inner cannula 178. Arms 174 may be, for example, permanent magnets and 2 mm long. Base 172 may have a sharp edge so as to facilitate secure placement in bone 182. Base 172 includes hinges 176 allowing arms 174 to pivotably move with respect to base 172. Hinges 176 bias arms 174 toward a position that is substantially perpendicular to inner cannula 178 (shown and described below with reference to FIG. 9). Hinges 176 also allow arms 174 to be positioned substantially parallel to inner cannula 178 as shown in FIG. 7. Second anchor 170 includes tines 180 disposed oblique to arms 174. Tines 180 may be, for example, 1-2 mm long.

FIG. 7 shows second anchor 170 being introduced in a vertebra 182 proximate to first anchor 130 and disc 110. Anchor 170 may be introduced in vertebra 182 using an introducer 184. Introducer 184 may generate a magnetic field pointing in the second direction, as shown by polarity marker 186. Introducer 184 may be, for example, a ferromagnetic material such as titanium. Arms 174 may generate a magnetic field pointing in the first direction, as shown by polarity marker 188. Introducer 184 thus attracts arms 174. Walls of outer sleeve 118, in combination with the magnetic attraction between introducer 184 and arms 180, cause arms 174 to move inwardly against the bias of hinge 176 toward position 174a as shown in FIG. 7.

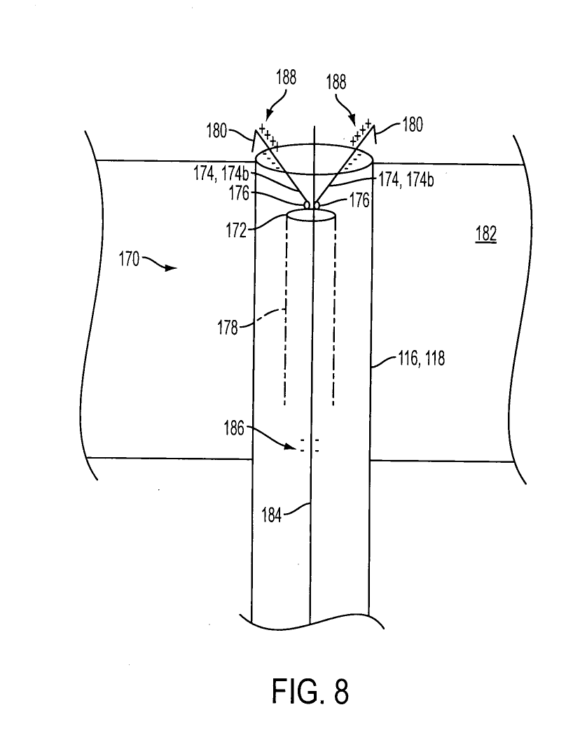

Referring to FIG. 8, a physician may continue to push second anchor 170 though outer sleeve 118 so that arms 174 extend beyond a periphery of outer sleeve 118. At this point, introducer 184 and arms 174 may be disposed in the foraminal canal and may be close to disc 110 (shown in prior figures). A physician may then start pulling introducer 184 back through outer sleeve 118. As arms 174 are no longer in proximity with the magnetic field generated by introducer 184, arms 174 start to move toward position 174b due to the bias of hinges 176. As the physician continues to pull back introducer 184, arms 174 continue to move outward and assume position 174c which is perpendicular to a length of inner cannula 178 as is shown in FIG. 9. The physician may continue to pull back on introducer 184 and inner cannula 178 to anchor tines 180 into vertebra 182. Thereafter, a screw 190 may be guided on introducer 184 through outer cannula 118 to attach to inner cannula 178 and secure second anchor 170 into vertebra 182. Screw 190 is designed to have a width wider than a width of outer sleeve 118. An outer wall of inner cannula 178 may be made of a porous material (illustrated by dashed and dotted line) so as to facilitate growth of vertebra 182 on to inner cannula 178. Introducer 184 may thereafter be removed from second anchor 170.

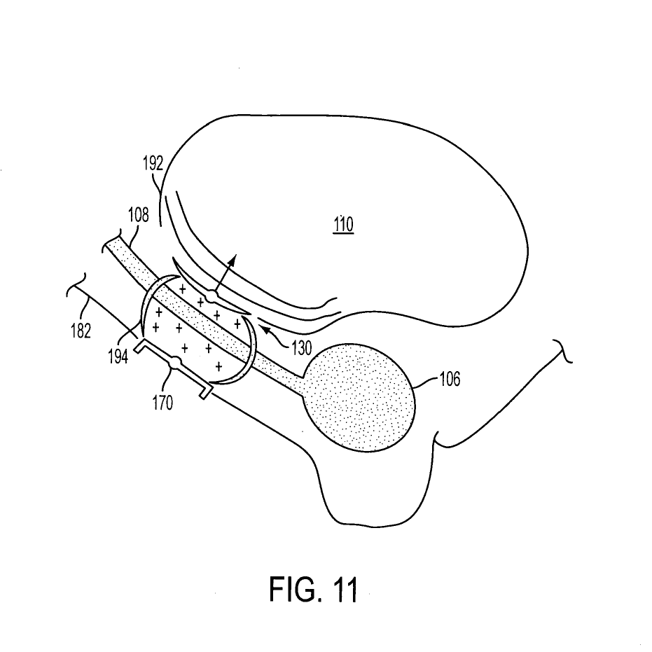

As shown in FIG. 11, first anchor 130 is anchored in disc 110 proximate to second anchor 170. As both first anchor 130 and second anchor 170 have magnetic fields pointing in the same direction, magnetic fields generated by first anchor 130 and second anchor 170 repel one another as shown by magnetic field marker 194. As second anchor 170 is implanted in bone 182, this repulsion causes a bulging portion 192 of disc 110 to be pushed back to its original location. Arms 140 of first anchor 130 and arms 174 of second anchor 170 may be designed to have varying magnetic field strengths. For example, a first set of magnets may generate a relatively weaker magnetic field than a second set of magnets which generate a relatively stronger magnetic field and more repulsion. Different sets of magnets may be used based on the clinical condition of the patient.

Unlike prior art devices and techniques, a spine surgery technique in accordance with the invention allows a physician to maintain the patient's anatomy and effectively push a bulging disc/herniated disc back into place away from a nerve. This results in less pain and recuperation time for the patient. In addition, the magnetic fields may be used to alleviate pain and other symptoms associated with degenerative disc disease.

The invention has been described with reference to an embodiment that illustrates the principles of the invention and is not meant to limit the scope of the invention. Modifications and alterations may occur to others upon reading and understanding the preceding detailed description. It is intended that the scope of the invention be construed as including all modifications and alterations that may occur to others upon reading and understanding the preceding detailed description insofar as they come within the scope of the following claims or equivalents thereof. Various changes may be made without departing from the spirit and scope of the invention. For example, although magnets are shown for each arm of the anchors, any number of magnets may be used such as one magnet per arm.

What is claimed is:

1. A method for performing spine surgery, the method comprising:
inserting a first anchor into a disc, the first anchor generating a first magnetic field pointing in a first direction, the disc being between a first and a second vertebra; and
inserting a second anchor into the first vertebra, the second anchor generating a second magnetic field pointing in the first direction.

2. The method as recited in claim 1, wherein the first anchor includes a first permanent magnet.

3. The method as recited in claim 1, wherein:
the first anchor comprises a first base and first and second arms pivotably connected to the first base; and
the first and second arms generate the first magnetic field.

4. The method as recited in claim 3, wherein:
the second anchor comprises a second base and third and fourth arms pivotably connected to the second base; and
the third and fourth arms generate the second magnetic field.

5. The method as recited in claim 4, wherein the first anchor further includes:
a body connected to the first base;
a hooked end connected to the body; and
wherein a first and second hinge pivotably connects the first and second arm to the base and further biases the first and second arms to a position substantially perpendicular to the body.

6. The method as recited in claim 5, wherein the inserting the first anchor comprises:
combining a first introducer with the first anchor, the first introducer having a third magnetic field pointing in a second direction opposite to the first direction;
moving the two arms of the first anchor toward the first introducer;
inserting the first anchor through the vertebra and into the disc; and
removing the first introducer from the first anchor.

7. The method as recited in claim 6, wherein the first anchor further comprises a first and a second tine disposed obliquely to the first and second arms of the first anchor.

8. The method as recited in claim 7, wherein the second anchor includes:
an inner cannula connected to the second base; and
wherein a third and fourth hinge pivotably connects the third and fourth arm to the second base and further biases the third and fourth arms to a position substantially perpendicular to the inner cannula.

9. The method as recited in claim 8, wherein the inserting the second anchor comprises:
combining a second introducer to the inner cannula, the second introducer having a fourth magnetic field pointing in the second direction opposite to the first direction;
moving the two arms of the second anchor toward the second introducer;
inserting the second anchor through the vertebra; and
removing the second introducer from the second anchor.

10. The method as recited in claim 9, wherein the second anchor further comprises a third and a fourth tine disposed obliquely to the third and fourth arms of the second anchor.

11. The method as recited in claim 10, further comprising screwing a screw to the inner cannula.

12. A method for performing spine surgery, the method comprising:
inserting a first cannula into a vertebra;
inserting a first anchor into the first cannula through the vertebra and into a disc, the first anchor generating a first magnetic field pointing in a first direction;
inserting a second anchor through the first cannula and through the vertebra, the second anchor generating a second magnetic field pointing in the first direction; and
pulling the second anchor into the vertebra.

13. The method as recited in claim 12, wherein:
the first anchor comprises a first base and first and second arms pivotably connected to the first base; and
the first and second arms generate the first magnetic field.

14. The method as recited in claim 13, wherein
the second anchor comprises a second base and third and fourth arms pivotably connected to the second base; and
the third and fourth arms generate the second magnetic field.

15. The method as recited in claim 14, wherein the first anchor further includes:
a body connected to the first base;
a hooked end connected to the body; and
wherein a first and second hinge pivotably connects the first and second arm to the base and further biases the first and second arms to a position substantially perpendicular to the body.

16. The method as recited in claim 15, wherein the inserting the first anchor comprises:
combining a first introducer with the first anchor, the first introducer having a third magnetic field pointing in a second direction opposite to the first direction;
moving the two arms of the first anchor toward the first introducer;
inserting the first anchor through the vertebra and into the disc; and
removing the first introducer from the first anchor.

17. The method as recited in claim 16, wherein the first anchor further comprises a first and a second tine disposed obliquely to the first and second arms of the first anchor.

18. The method as recited in claim 17, wherein the second anchor includes:
an inner cannula connected to the second base; and
wherein a third and fourth hinge pivotably connects the third and fourth arm to the second base and further biases the third and fourth arms to a position substantially perpendicular to the inner cannula.

19. The method as recited in claim 18, wherein the inserting the second anchor comprises:
combining a second introducer to the inner cannula, the second introducer having a fourth magnetic field pointing in a second direction opposite to the first direction;
moving the two arms of the second anchor toward the second introducer;
inserting the second anchor through the vertebra; and
removing the second introducer from the second anchor.

20. The method as recited in claim 19, wherein the second anchor further comprises a third and a fourth tine disposed obliquely to the third and fourth arms of the second anchor.

21. The method as recited in claim 17, further comprising screwing a screw to the inner cannula.

22. A system for use in spine surgery, the system comprising:
a first anchor, the first anchor including
a first base, and
first and second arms pivotably connected to the first base, the first and second arms generating a first magnetic field pointing in a first direction, and
a second anchor, the second anchor including
a second base, and
third and fourth arms pivotably connected to the second base, the third and fourth arms generating a second magnetic field pointing in the first direction.

23. The system as recited in claim 22, further comprising:
a first and a second tine disposed obliquely to the first and second arms; and
a third and a fourth tine disposed obliquely to the third and fourth arms.

24. The system as recited in claim 23, wherein the first anchor further includes:
a body connected to the first base;
a hooked end connected to the body; and wherein
a first and second hinge pivotably connects the first and second arm to the first base and further biases the first and second arms to a position substantially perpendicular to the first base.

25. The system as recited in claim 24, wherein the second anchor further includes:
an inner cannula connected to the second base; and wherein
a third and fourth hinge pivotably connects the third and fourth arm to the second base and further biases the third and fourth arms to a position substantially perpendicular to the inner cannula.

26. The system as recited in claim 25, further comprising;
an introducer, the introducer generating a magnetic field pointing in a second direction opposite the first direction; and wherein
the body includes a notch effective to receive the introducer.

27. The system as recited in claim 26, further comprising a screw effective to screw to the inner cannula.
